(19) United States
(12) Patent Application Publication
(10) Pub. No.: US 2005/0143650 A1
(43) Pub. Date: Jun. 30, 2005

(54) Tissue marker and method and apparatus for deploying the marker

(76) Inventor: Axel Winkel, Schwerin (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/996,754

(22) Filed: Nov. 24, 2004

Related U.S. Application Data
(60) Provisional application No. 60/525,205, filed on Nov. 26, 2003.

(51) Int. Cl. A61B 5/05

(52) U.S. Cl. 600/423

(57) ABSTRACT

The subject invention pertains to a tissue marker. The subject invention also relates to methods and apparatus for deploying a tissue marker. In a specific embodiment, the subject marker is magnetic resonance imaging (MRI) compatible. In additional embodiment, various components of the subject marker deploying apparatus are MRI compatible. A specific marker in accordance with the subject invention is flexible such that the marker has an equilibrium shape, which the marker will have when deployed in tissue to be marked, and an elongated shape, which the marker can be bent into to be inserted into a marker needle.

### CROSS-REFERENCE TO RELATED APPLICATIONS

This application claims the benefit of provisional patent application Ser. No. 60/525,205, filed Nov. 26, 2003, which is hereby incorporated by reference in its entirety.

### TECHNICAL FIELD

The present invention relates in general to a tissue marker system, a method of deploying a tissue marker, and a tissue marker. A specific embodiment of the subject marker is compatible with magnetic resonance imaging (MRI) and is visible under MRI. In another embodiment, various components of the subject marker system are compatible with MRI such that the marker can be deployed under MRI.

### BACKGROUND

Tissue markers are used in medicine to mark a suspicious lesion for surgical planning or for follow-up diagnostics. Markers are placed or deployed into tissue and remain in the tissue for a period of time. In order for markers not to move, markers are often clamped or stapled into tissue to hold them in place. Such markers which are clamped or stapled into tissue are sometimes called clips. In surgical planning, clips can be used to mark an area to be extracted. In follow-up diagnostics clips can be used to mark a suspicious lesion for long-term surveillance. Several clips are known from the literature.

U.S. Pat. No. 6,261,302 discloses an applier for initially delivering a biopsy marker to a surgical biopsy site and subsequently deploying the marker at the site. The applier includes an elongated flexible tube having a distal end; a ferrule fixed to the distal end of the flexible tube, the ferrule having a forming surface thereon adapted to reconfigure the biopsy marker from an original open configuration to a closed configuration when the biopsy marker has been delivered to the surgical site; and a marker holder at the ferrule for holding the biopsy marker at the distal end of the flexible tube in the original open position.

U.S. Pat. No. 6,228,055 discloses a device for marking a particular tissue area. The device includes: a discrete marker element and an apparatus for remotely delivering the marker element from outside the human body to the particular tissue area, using an aided visualization device wherein the device is adapted to be employed in combination with a medical instrument which transports the device to the selected tissue location and draws a vacuum to isolate and retain tissue at the selected location.

U.S. Ser. No. 09/776,125 (U.S. Patent Application Publication No. 2001/0034528) discloses a device for marking a particular tissue, which includes an apparatus including a member being adapted to receive a deployment actuator connector, where the deployment actuator connector has a predetermined failure point in the distal region of the deployment actuator connector such that the proximal portion of the deployment actuator connector being adapted to be severed from the distal portion at the predetermined failure point upon further activation of the deployment actuator after abutment of the marker element against a stop designed into the member distal region.

U.S. Pat. No. 5,989,265 discloses a device for pinpointing lesions detected in a breast and an apparatus for positioning a pinpointing device. The device includes an anchor and at least one wire different from and attached to the anchor and of sufficient length for implantation of the anchor within the breast and includes an anchor having a plurality of flexible and elastic strands and a wire different from and attached to the strands at first ends of the strands and of sufficient length for implantation of the anchor within the breast. The apparatus includes a pinpointing device and at least one wire different from and attached to the anchor and of sufficient length for implantation of the anchor within the breast.

U.S. Pat. No. 5,902,310 discloses a method and apparatus for marking a particular tissue area, involving a marking apparatus, a marker element applier, or a method of marking tissue, each incorporating a pull wire or pulling on a pull wire. The apparatus includes a pull wire having a distal end and a proximal end, the marker element being attached to the distal end of the pull wire.

U.S. Pat. No. 6,511,498 discloses a surgical device for anchoring a thread or wire to a bone having a hole bored therein. The surgical device includes a deformable tubular sleeve which is capable of deforming between a first stretched position of low cross section and a second folded position of greater cross section, and a thread whose middle part is in the form of a closed loop passing through the deformable tubular sleeve. The deformable tubular sleeve is able to slide on the thread within the limits of the closed loop.

U.S. Pat. No. 6,425,903 discloses an implantable marker for implantation of tissue of a surgical patient. The implantable marker has a base, a first leg including a first camming marker surface, a second leg including a second camming marker surface, and a first camming marker surface notch located on the first camming marker surface and a second camming marker surface notch located on the second camming marker surface.

U.S. application Ser. No. 10/028,753 (U.S. Patent Application Publication No. US2002/0083951) discloses an implantable identification marker and a method for implanting an identification marker including an electronic device enclosed within a biocompatible material isolating the device from body fluids of the animal.

U.S. Pat. No. 5,941,890 discloses an implantable marker for implantation of tissue of a surgical patient. The implantable marker has a base, a first leg including a first camming marker surface, a second leg including a second camming marker surface, and a first reverse cleat protruding from the first camming marker surface and a second reverse cleat protruding from the second camming marker surface.

These clips can lose their position if unintentionally loosened or the mechanism used to attach to the tissue is disturbed. There is a need for a marker which can hold its position in tissue even when the marker is disturbed or loosened. There is also a need for a method and an apparatus for deploying a marker which can hold its position in tissue even when the marker is disturbed or loosened.

### BRIEF SUMMARY OF INVENTION

The subject invention pertains to a tissue marker. The subject invention also relates to methods and apparatus for deploying a tissue marker. In a specific embodiment, the subject marker is magnetic resonance imaging (MRI) compatible. The subject tissue marker can be visible under CT and/or ultrasound. In additional embodiment, various components of the subject marker deploying apparatus are MRI compatible. A specific marker in accordance with the subject invention is flexible such that the marker has an equilibrium shape, which the marker will have when deployed in tissue to be marked, and an elongated shape, which the marker can be bent into to be inserted into a marker needle.

In a specific embodiment, the subject invention utilizes a freestanding marker clip, or marker, which is released by pushing the marker out of the end of an insertion tube, or marker needle. Advantageously, the freestanding marker is not attached to anything protruding from the patient once the marker is positioned. In a specific embodiment, the subject invention involves pushing a marker out of the distal end of an insertion tube with a plunger, or ejecting rod. Although, the ejecting rod can be flexible the ejecting rod is sufficiently stiff to push the marker out of the marker needle. In a specific embodiment, the marker employed by subject invention is a circular ring shaped marker, which can be elongated in order to be positioned within the insertion tube.

To mark the tissue, the subject clip can be positioned next to a lesion like a tag. A marker having a bigger diameter than the lesion can be used and can surround the lesion in one or more dimensions like a cage. This cage can describe the position and size of the lesion and can, if desired, include a safety-margin. Surrounding the lesion makes the surgical or minimal invasive procedure to take out or destroy the lesion much more effective, as the size, position, and shape of the lesion can be discerned by the size, position, and shape of one or more markers used to appropriately mark the lesion.

The subject method and apparatus for deploying a marker can utilize a plurality of markers that can be identified with respect to each other. In an embodiment, two or more markers that can be distinguished from each other can be deployed. Such markers can be differentiated from each other via, for example, the size of the markers, the shape of the markers, and/or the characteristics of the markers under MRI or other imaging modality. The subject clip can also be modified to improve visibility of the marker under ultrasound. In specific embodiments, the subject marker can be coated with chemicals and/or bioactive materials. Such chemicals and/or bioactive materials can impact the therapy provided to the patient.

In a specific embodiment, the subject marker can be modified to enhance visibility under MRI. Such modification can include the incorporation of a microcoil with the marker that can be imaged under MRI such that the marker can be found much faster under MRI than without the microcoil. In an embodiment, the microcoil can be incorporated in an implantable plastic capsule that is implanted in the marker.

### BRIEF DESCRIPTION OF THE DRAWINGS

The invention may be more completely understood in consideration of the following detailed description of various embodiments of the invention in connection with the accompanying drawings, in which:

FIG. 1 schematically illustrates the shape of a marker in accordance with the subject invention, when deployed correctly in human or animal tissue;

FIG. 2A schematically illustrates a cross sectional view of a marker deployment assembly in accordance with the subject invention, having a marker, a cannula, and an ejecting device;

FIG. 2B schematically illustrates an enlarged view of the tip of FIG. 2A;

FIG. 3A schematically illustrates a marker deployment assembly in accordance with the subject invention, as a marker is being ejected.

FIG. 3B schematically illustrates the marker deployment assembly of FIG. 3A, when the marker is almost fully ejected.

FIGS. 4A-4E schematically illustrate a variety of different marker shapes in accordance with the subject invention, where FIG. 4A shows a marker having a flexible portion 11 and extension portion 50, which can vary for each of a plurality of markers in order to differentiate the markers from each other; FIG. 4B shows the marker of FIG. 4A having an additional flexible portion extending from the other end of extension portion 50; FIG. 4C shows a marker having identifying portions 51 of the flexible portion 11 which can vary for each of a plurality of markers in order to differentiate the markers from each other; FIG. 4D shows a marker having a plurality of extensions extending from one end of the flexible portion 11 so as to enhance the visibility of the marker under ultrasound; and FIG. 4E shows a marker having an extension extending from flexible portion 11, where the extension has one or more sections where the marker is bunched up.

FIGS. 5A-5B show a marker having a flexible portion 11 and a modified portion 55, which is coated and/or incorporates chemicals or other substances that can migrate from the modified portion to the surrounding tissue after deployment of the marker, where FIG. 5A shows the marker prior to deployment and FIG. 5B shows the marker after deployment and migration of the chemical or other substance from the modified portion of the marker into the tissue.

FIG. 6 schematically illustrates a marker in accordance with the subject invention deployed proximate a lesion 58 so as to form a cage around the lesion.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

The subject invention relates to methods and apparatus for positioning a tissue marker in human or animal tissue. The subject invention also pertains to a tissue marker. Preferably, the subject tissue markers are visible under one or more imaging modalities such as, but not limited to, x-ray, ultrasound, and magnetic resonance imaging (MRI). In a specific embodiment, the subject marker is visible under magnetic resonance imaging (MRI).

In a specific embodiment, the subject invention incorporates a marker needle, an ejecting rod, and a tissue marker. The marker needle can penetrate the tissue to be marked such that the distal tip of the marker needle is positioned at the tissue location to be marked. The tissue marker can be inserted into the marker needle so as to be in an elongated position inside the marker needle. The marker can be inserted into the marker needle before or after positioning the marker needle. In a preferred embodiment, the marker is inserted into the marker needle prior to the marker needle penetrating the tissue to be marked. Once the marker needle is positioned such that a distal end of the marker needle is at the site to be marked by the tissue marker and the marker is inserted into the marker needle, the ejecting rod can slide within the marker needle and push the tissue marker out the distal end of the marker needle. The tissue marker returns to its equilibrium shape as it is pushed out of the marker needle. The tip of the marker is shaped, and the body of the marker is designed, so that the marker circumvents the tissue to be marked as the marker returns to its equilibrium shape while being ejected out of the marker needle. In a specific embodiment, the tip of the tissue needle is shaped to that the tissue marker penetrates the surrounding tissue as it is pushed out of the marker needle and continues to penetrate the tissue as the marker returns to its equilibrium shape and is pushed entirely out of the marker needle. The tissue marker is then securely positioned at the desired site in the human or animal tissue and can be used to locate the site under, for example, MRI.

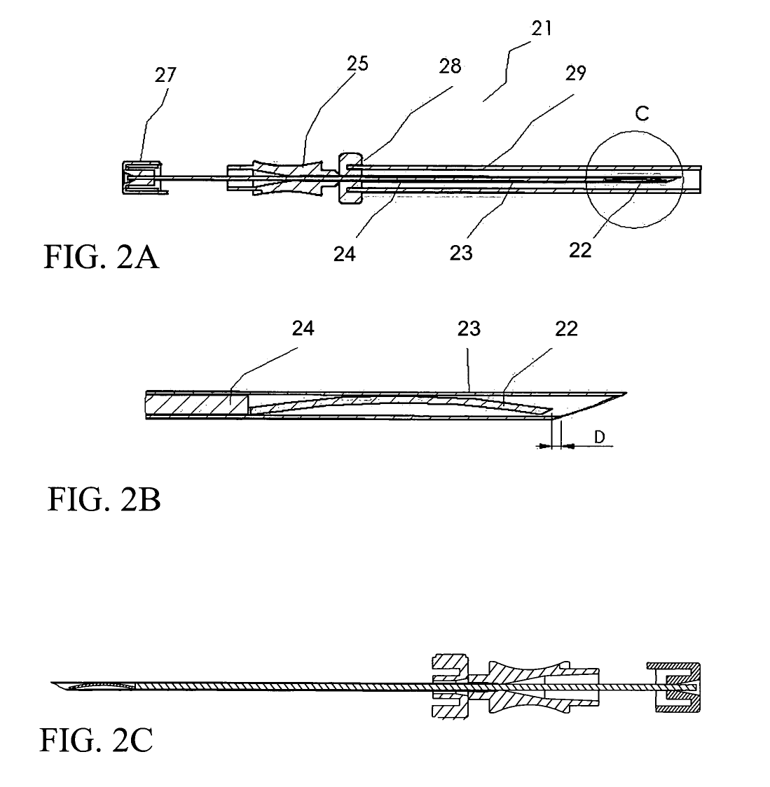

In another specific embodiment, the subject marker apparatus can be adapted such that the marker needle, or insertion tube, can be inserted into a guidance tube which is positioned in the tissue. The distal end of the additional guidance tube can be positioned near the location to be marked such that the marker needle is inserted into the guidance tube and can deploy the marker out the distal end of the guidance tube. In this embodiment, the marker needle and ejecting rod can be flexible enough to follow the path of the guidance tube, which need not be straight. In addition, the distal tip of the marker needle can be flat, or have an alternative shape which may assist the deployment of the marker. In contrast, referring to FIG. 2B, the distal tip of a marker needle which penetrates tissue is preferably pointed to allow easier penetration of tissue, although other distal tip shapes can be used as well.

With one or more clips, or markers, of different sizes and/or shapes it is possible to create a 3 dimensional cage around the lesion that can include a safety margin for the surgical or minimally invasive treatment. This can provide much better control of the tissue that is taken out and can allow a caregiver to work with very small safety margins without leaving tumor behind. The blades of the cutting device can be guided by the structure of the clip-cage.

The subject method and apparatus for deploying a marker can utilize a plurality of markers that can be identified with respect to each other. For example, two or more markers that can be distinguished from each other can be deployed. Such markers can be differentiated from each other via, for example, the size of the markers, the shape of the markers, and/or the characteristics of the markers under MRI or other imaging modality. The subject clip can also be modified to improve visibility of the marker under ultrasound. In specific embodiments, the subject marker can be coated with chemicals, bioactive materials, and/or other substances. Such chemicals, bioactive materials, and/or other substances can impact the therapy provided to the patient.

In a specific embodiment, the subject marker can be modified to enhance visibility under MRI. Such modification can include the incorporation of a microcoil with the marker that can be imaged under MRI such that the marker can be found much faster under MRI than without the microcoil. In an embodiment, the microcoil can be incorporated in an implantable plastic capsule that is implanted in the marker.

In another specific embodiment employing at least two markers of different sizes, a first marker can be positioned around a lesion of a patient and one or more small clips can be positioned so as to surround the lesion, allowing the surgeon to remove the lesion with a margin of safety.

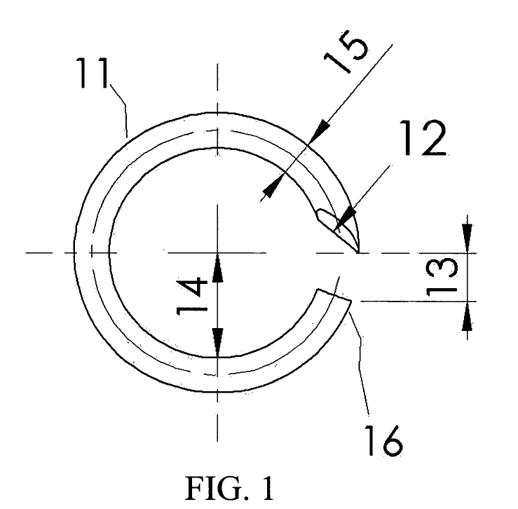

FIG. 1 shows one embodiment of the subject tissue marker 11. In this embodiment, the subject tissue marker 11 is a circular shaped solid or hollow needle. The marker needle can be made from a variety of materials, including, but not limited to, a titanium alloy such as T13A12.54, stainless steel, other titanium alloys, or plastic. A specific stainless steel which can be used is 316L ASTM. Another specific titanium alloy that can be used is titanium-nickel, NITI SE 508. Specific examples of plastics which can be used are PE, PP, PU, PEEK, Teflon and PEI. Contrast enhancing substances can be incorporated in the plastic material, such that the plastic tissue marker is visible under MRI. Examples of contrast enhancing substances which can be used include gadolinium particles for a positive contrast and metallic particles for a negative contrast. In a specific embodiment, particles can range in size from about 10 to about 100 micrometers.

As shown in FIG. 1, the marker 11 is circularly shaped. In a specific embodiment, the marker can have a radius 14 between about 2 and about 30 mm, and preferably about 5 mm. The equilibrium shape of the marker 11 can be such that the marker 11 can be elongated to be inserted within the marker needle and, as pushed out of the marker needle by the ejecting rod, the marker penetrates the surrounding tissue as it returns to its equilibrium shape. In a specific embodiment, the marker tip penetrates the surrounding tissue as the marker is ejected from the marker needle such that the rest of the marker body follows the path of the marker tip. Once the marker is completely ejected from the marker needle, the marker is securely attached to the surrounding tissue. Preferably, the marker is circular but other shapes can be used as well. Examples of other equilibrium shapes the marker can have include, but are not limited to, ellipsoidal and oval, not shown here. The marker can lie in a plane, as shown in FIG. 1, or can lie on, for example, a helical path. Other geometric contours can be utilized for the marker design.

In a specific embodiment, the diameter 15 of the body portion of the solid or hollow marker can be between about 0.1 mm and about 1.5 mm, and is preferably about 0.35 mm. The marker can be manufactured from a wire with different cross-sectional shapes, such as round, square, or twisted. The wire can be solid or hollow. In another specific embodiment, the marker can be cut from a tubular piece of material having an appropriate diameter and cross-sectional shape, such that the marker has the desired mechanical properties. In a specific embodiment, the circular marker 11 does not quite close to a circular loop, but has an opening 13. In a specific embodiment, this opening can be between about 1 mm and about 5 mm, preferably about 1.5 mm. The marker can have a distal tip 12 and a proximal tip 16. The distal tip 12 should be sharp enough to pierce the surrounding tissue as the marker is ejected from the marker needle and the proximal tip 16 should be shaped such that the ejecting rod can contact and push the proximal tip 16 of the marker so as to push the marker out of the marker needle. In a preferred embodiment, the proximal tip is blunt such that the ejecting rod can push the marker out of the marker needle without binding the marker against the side of the marker needle.

FIG. 2A illustrates a cross sectional side view of a marker deployment assembly 21 in accordance with the subject invention. The marker deployment assembly shown in FIG. 2A includes an elongated marker 22, which is an elongated version of marker 11 of FIG. 1; ejecting rod 24 with handle 27; marker needle 23 with handle 25; and an optional protection cover 29. The protection cover 29 with proximal adapter 28 protects the assembly 21 when stored or shipped and is removed before the medical marking procedure starts. Adapter 28 adapts to the handle 25 of marker needle 23. FIG. 2B is an enlarged view of the tip of the assembly 21. Tissue marker 22 is stretched within its elasticity limits from its circular ground shape 11, as seen in FIG. 1, to an elongated form as seen in FIG. 2B. Marker needle 23 holds the marker 21 in its elongated form. At its blunt proximal end the marker contacts the ejecting rod 24. In a specific embodiment, the marker needle 23 has an outer diameter of between 0.5 mm to about 2 mm, and preferably about 1.3 mm. In a specific embodiment, the wall thickness of the marker needle 23 is typically between about 0.1 mm and about 0.3 mm. In a specific embodiment, prior to insertion of the marker needle into the tissue to be marked, the distal tip of the marker 23 can be positioned such that the distance D between the opening of the distal tip of the marker needle 23 and the distal tip of the marker 23 is between about 0.5 mm and about 8 mm, and preferably about 3 mm. Again, protective cover 29 is optional and can be used to protect marker needle 23 when not being inserted into tissue to be marked.

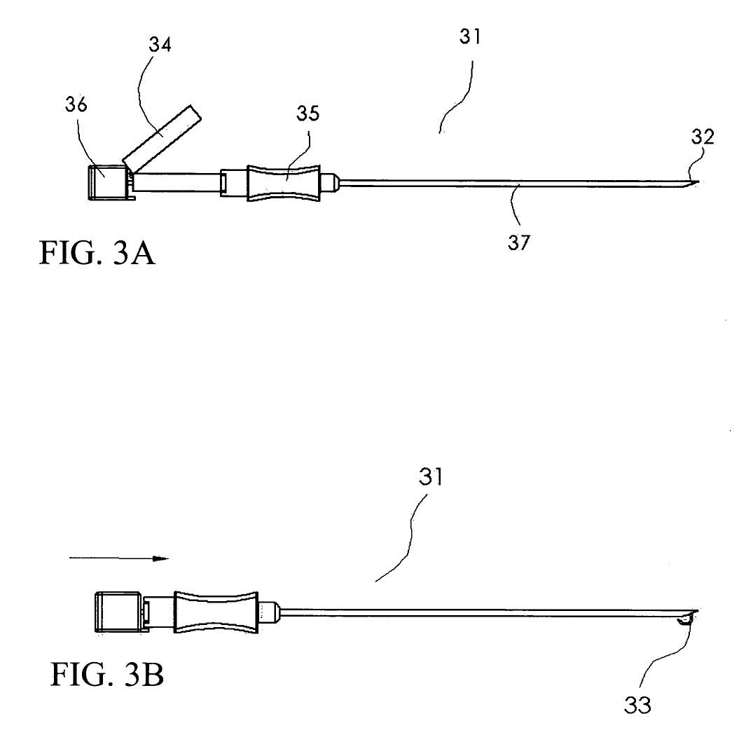

FIG. 3 illustrates an embodiment of the subject invention during use. Marker assembly 31 (without protection cover, not shown in this figure) is injected into the body tissue and penetrated to the location in which the marker 33 is to be deployed. The location can be controlled by radiological standard modalities, such as, but not limited to, x-ray, ultrasound, and/or MRI. Once the marker needle is positioned such that the marker will be in the desired location upon ejection from the marker needle, an optional deployment protection tube 34 can be removed. When employed, this protection tube 34 can prevent the handle 35 of marker needle 37 from moving towards the handle 36 of the ejection rod so as to prevent ejection, or partial ejection, of the marker while positioning of marker needle 37 in the tissue. Other means can also be utilized to reduce the occurrence of ejecting or partially ejecting the marker prior to final positioning of the marker needle. Once the deployment protection tube 34, or other means for limiting movement of the marker within the marker needle, is disengaged, the operator of the marker assembly can grasp handle 35 of the marker needle 37 and push the handle 36 of the ejection rod towards handle 35, while holding handle 35 still (see arrow).

As shown in FIG. 2 and FIG. 3, the ejection rod 24 pushes the marker 22 out of the marker needle 23. As shown in FIG. 3, as the marker is ejected from the distal tip of the marker needle, the marker returns to its equilibrium shape, which in the embodiment shown in FIG. 3 is circular. While being pushed forward, the marker 33 penetrates through tissue and circumvents tissue. Hence, to stay in its location, the subject marker does not clamp tissue, like clips typically do, but stays by circumventing tissue. Once the marker 33 is fully pushed out, or deployed, the needle assembly can be pulled backwards and removed from the tissue, leaving the marker embedded in the tissue.

The subject needle assembly can have one or more optional features, such as a means to suction out air or blood before ejecting the marker to enhance the likelihood that the marker is fixed to tissue and not, for example, loose inside a cavity. Circumventing the tissue can reduce, or prevent migration of the marker with respect to the tissue after placement of the marker. If the distal tip 12 of the marker is sufficiently sharp to allow piercing of the tissue by the marker tip, migration of the marker with respect to the tissue can be further reduced, or prevented.

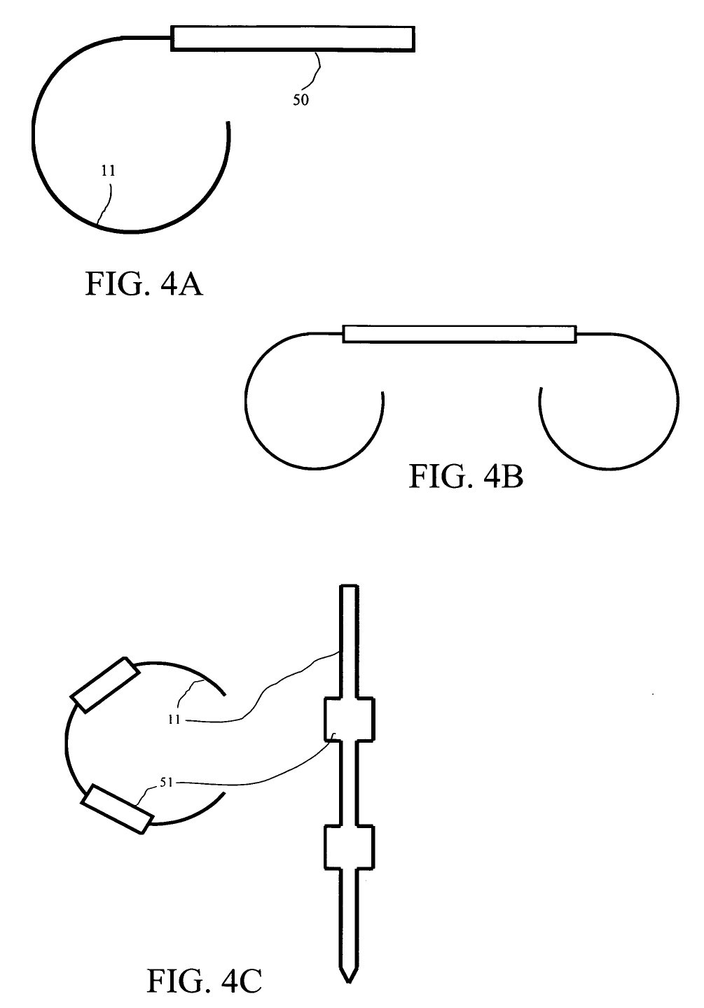

FIGS. 4A-4E schematically illustrate a variety of different marker shapes in accordance with the subject invention, where FIG. 4A shows a marker having a flexible portion 11 and extension portion 50, which can vary for each of a plurality of markers in order to differentiate the markers from each other; FIG. 4B shows the marker of FIG. 4A having an additional flexible portion extending from the other end of extension portion 50; FIG. 4C shows a marker having identifying portions 51 of the flexible portion 11 which can vary for each of a plurality of markers in order to differentiate the markers from each other; FIG. 4D shows a marker having a plurality of extensions extending from one end of the flexible portion 11 so as to enhance the visibility of the marker under ultrasound; and FIG. 4E shows a marker having an extension extending from flexible portion 11, where the extension has one or more sections where the marker is bunched up. The shape and size of extension portion 50, identifying portions 51, extensions, and bunched up wire sections are not necessarily drawn to scale. The markers shown in FIG. 4B can be employed by a first flexible portion 11 exiting the marker needle, then the extension portion, and finally the other flexible portion. The marker shown in FIG. 4C can be, for example, cut from a tube and identifying portions 51 can be integral with flexible portion 11. The marker shown in FIG. 4E can be positioned in the needle and weaker section can bunch up upon pushing the marker with the ejecting rod.

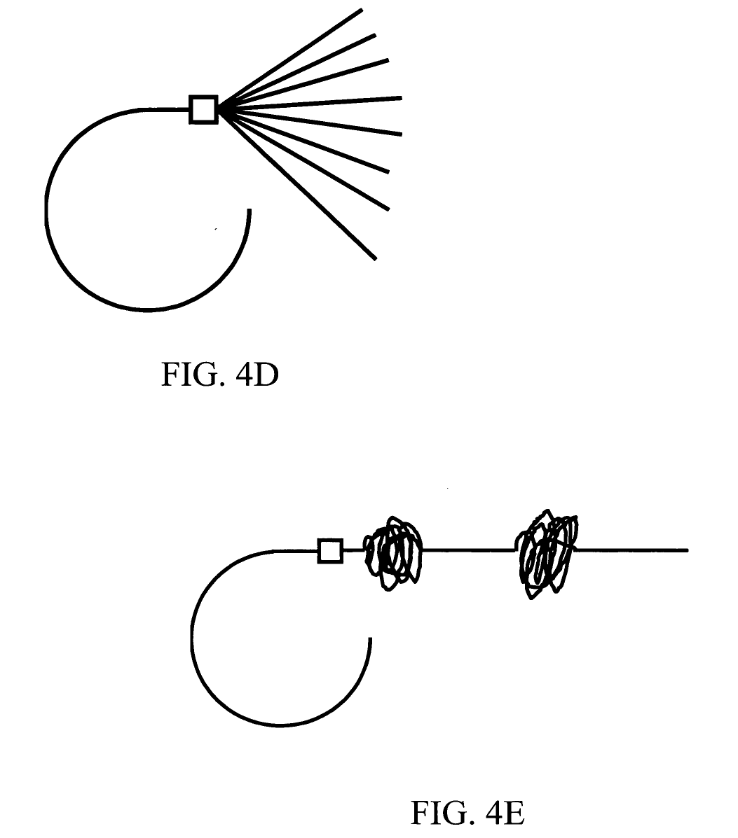

FIGS. 5A-5B show a marker having a flexible portion 11 and a modified portion 55, which is coated and/or incorporates chemicals or other substances that can migrate from the modified portion to the surrounding tissue after deployment of the marker, where FIG. 5A shows the marker prior to deployment and FIG. 5B shows the marker after deployment and migration of the chemical or other substance from the modified portion of the marker into the tissue.

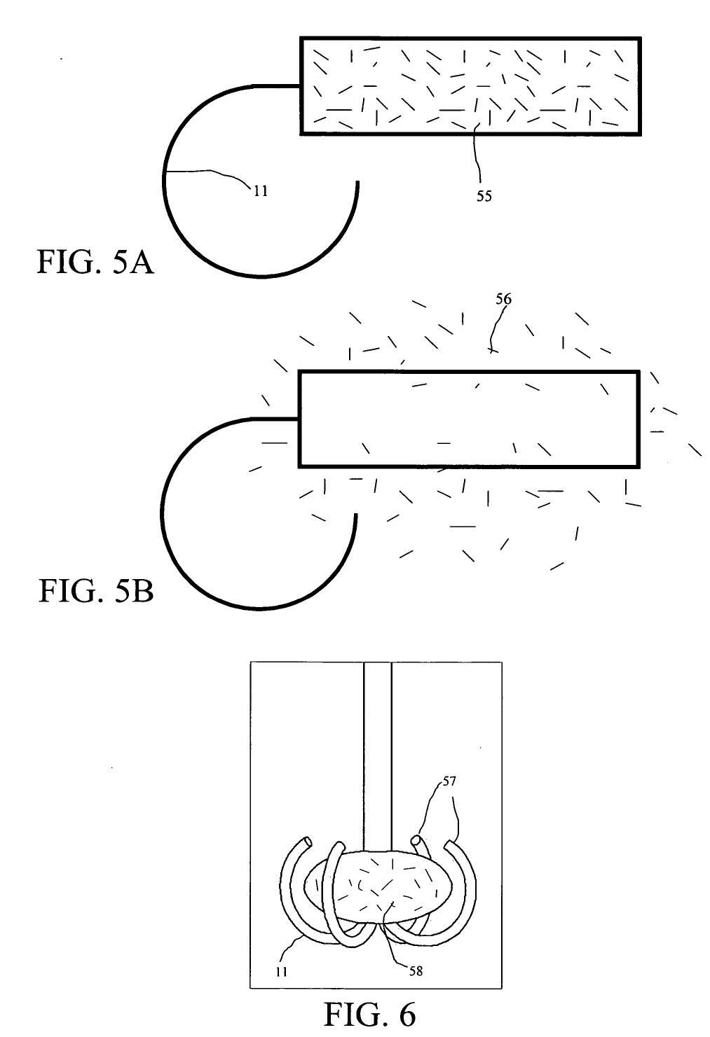

FIG. 6 schematically illustrates a marker in accordance with the subject invention deployed proximate a lesion 58 so as to form a cage around the lesion. The cage can be formed by a plurality of individual markers deployed sequentially, or simultaneously, or by a single marker having a plurality of flexible portions 11 and 57.

The subject invention can utilize needles which are disposable or reusable. In a specific embodiment, various markers can be re-load in the subject marker needle.

While the above description of the invention has been presented in terms of a human subject (patient), it is appreciated that the invention may also be applicable to treating other subjects, such as mammals, organ donors, cadavers and the like.

The present invention should not be considered limited to the particular embodiments described above, but rather should be understood to cover all aspects of the invention as fairly set out in the appended claims. Various modifications, equivalent processes, as well as numerous structures to which the present invention may be applicable will be readily apparent to those skilled in the art to which the present invention is directed upon review of the present specification. The claims are intended to cover such modifications and devices.

To improve the visibility of the clips for different imaging modalities the subject marker can be MRI compatible and can incorporate a structure that can be visualized under CT and/or ultrasound as well. For example, a plastic marker in accordance with the subject invention can incorporate some metal to be visible under CT. In an embodiment, referring to FIG. 4E, one or more bunched wire portions can be used or, referring to FIG. 4D, a wire fan can be used to improve visibility for ultrasound. Collagen ultrasound markers can also be incorporated. The number, shape, and size of these portions can be varied to differentiate the clips from each other.

In another embodiment, chemicals can be incorporated with the marker to enhance visibility. For ultrasound visibility a coating with contrast agent (microbubbles) can be used. By using a biocompatible chemical substance to color the tissue, such as potassium permanganate, around the clip it is much easier for the surgeon to find the clip during the operation because the colored area is bigger than the clip. By using such a “color eluting clip” the surgeon can find the clip very easily because the tissue around the clip is colored.

The subject clip, or marker, can also carry chemicals that have direct therapeutic effects or make a minimal invasive treatment more effective.

1. An apparatus for deploying a tissue marker, comprising:
a marker needle having hollow portion for receiving a tissue marker, wherein the tissue marker has an equilibrium shape and an elongated shape, wherein once the tissue marker is received by the hollow portion of the marker needle the tissue marker is in its elongated shape, and
an ejecting rod, wherein the ejecting rod slides within the marker needle to push the tissue marker out of a distal end of the marker needle,
wherein the tissue marker forms the elongated shape upon insertion of the tissue marker into the hollow portion of the marker needle and returns to the equilibrium shape upon ejection from the marker needle.

2. The apparatus according to claim 1, wherein the distal end of the marker needle is pointed for penetrating tissue.

3. The apparatus according to claim 1, further comprising a guidance tube, wherein the marker needle slides within the guidance tube such that the distal end of the marker needle extends out a distal end of the guidance tube in order to eject the tissue marker from the marker needle.

4. The apparatus according to claim 3, wherein the marker needle is flexible and the ejecting rod is flexible, wherein the marker needle and the ejecting rod follow the shape of the guidance tube.

5. The apparatus according to claim 3, wherein the guidance tube is straight.

6. The apparatus according to claim 3, wherein the guidance tube is curved.

7. The apparatus according to claim 3, wherein the distal end of the marker needle is flat.

8. The apparatus according to claim 1, further comprising a means for suctioning air or blood from a cavity.

9. The apparatus according to claim 1, further comprising a means for limiting movement of the tissue marker within the marker needle.

10. The apparatus according to claim 9, wherein the means for limiting movement of the tissue marker within the marker needle comprises a deployment protection tube, wherein the deployment protection tube prevents a handle of the marker needle from moving towards a handle of the ejection rod such that the tissue marker is prevented from ejecting during positioning of the marker needle.

11. The apparatus according to claim 1, further comprising a protection cover for receiving the marker needle.

12. The apparatus according to claim 1, wherein the marker needle and ejecting rod are MRI compatible.

13. The apparatus according to claim 1, wherein the equilibrium shape of the tissue marker is substantially circular.

14. An apparatus for locating a tissue marker, comprising:
a tissue marker, the tissue marker having an equilibrium shape and an elongated shape,
a marker needle having hollow portion for receiving a tissue marker, wherein the tissue marker has an equilibrium shape and an elongated shape, wherein once the tissue marker is received by the hollow portion of the marker needle the tissue marker is in its elongated shape, and
an ejecting rod, wherein the ejecting rod slides within the marker needle to push the tissue marker out of a distal end of the marker needle,
wherein the tissue marker forms the elongated shape upon insertion of the tissue marker into the hollow portion of the marker needle and returns to the equilibrium shape upon ejection from the marker needle.

15. The apparatus according to claim 14, wherein the distal end of the marker needle is pointed for penetrating tissue.

16. The apparatus according to claim 14, further comprising a guidance tube, wherein the marker needle slides within the guidance tube such that the distal end of the marker needle extends out a distal end of the guidance tube in order to eject the tissue marker from the marker needle.

17. The apparatus according to claim 16, wherein the marker needle is flexible and the ejecting rod is flexible, wherein the marker needle and the ejecting rod follow the shape of the guidance tube.

18. The apparatus according to claim 16, wherein the guidance tube is straight.

19. The apparatus according to claim 16, wherein the guidance tube is curved.

20. The apparatus according to claim 16, wherein the distal end of the marker needle is flat.

21. The apparatus according to claim 14, further comprising a means for suctioning air or blood from a cavity.

22. The apparatus according to claim 14, further comprising a means for limiting movement of the tissue marker within the marker needle.

23. The apparatus according to claim 22, wherein the means for limiting movement of the tissue marker within the marker needle comprises a deployment protection tube, wherein the deployment protection tube prevents a handle of the marker needle from moving towards a handle of the ejection rod such that the tissue marker is prevented from ejecting during positioning of the marker needle.

24. The apparatus according to claim 14, further comprising a protection cover for receiving the marker needle.

25. The apparatus according to claim 14, wherein the marker needle and ejecting rod are MRI compatible.

26. The apparatus according to claim 14, wherein the equilibrium shape of the tissue marker is substantially circular.

27. The apparatus according to claim 14, wherein the tissue marker is MRI compatible.

28. The apparatus according to claim 14, wherein the tissue marker comprises an extension portion which remains substantially straight when the tissue marker is inserted in the marker needle and after ejection from the marker needle.

29. The apparatus according to claim 14, wherein the tissue marker comprises a microcoil visible under MRI.

30. The apparatus according to claim 14, wherein the tissue marker comprises a titanium alloy.

31. The apparatus according to claim 14, wherein the tissue marker comprises stainless steel.

32. The apparatus according to claim 14, wherein the tissue marker comprises plastic.

33. The apparatus according to claim 14, wherein the tissue marker comprises an MRI contrast enhancing substance.

34. The apparatus according to claim 14, wherein the equilibrium shape of the marker is substantially circular.

35. The apparatus according to claim 34, wherein the tissue marker has a radius between about 2 mm and about 30 mm.

36. The apparatus according to claim 14, wherein the tissue marker has a pointed distal end, wherein the distal end of the tissue marker exits the distal end of the marker needle first upon ejection of the tissue marker.

37. The apparatus according to claim 34, wherein the equilibrium shape of the tissue marker forms a circular loop with an opening in the circular loop.

38. The apparatus according to claim 14, wherein the tissue marker comprises a plurality of extensions which enhance the tissue marker's visibility under ultrasound.

39. The apparatus according to claim 14, wherein the tissue comprises a chemical which migrates into the surrounding tissue after deployment of the tissue marker.

40. The apparatus according to claim 14, wherein the tissue marker comprises a bioactive material which migrates into the surrounding tissue after deployment of the tissue marker.
